(19) United States
(12) Patent Application Publication
(10) Pub. No.: US 2023/0352146 A1
(43) Pub. Date: Nov. 2, 2023

(54) SYSTEMS AND METHODS FOR OPTIMAL PERSONALIZED INFANT NUTRITION, DISEASE PREVENTION, AND GROWTH MONITORING

(71) Applicant: TELLSPEC, LTD., London (GB)

(72) Inventors: Isabel De Oliveira Correa, London (GB); Diogo Barros, London (GB); Paolo Satta, London (GB); Rafael Queirós, London (GB); Luis Miguel Fernandes Pires, London (GB); Mark Bloore, London (GB)

(73) Assignee: TELLSPEC, LTD., London (GB)

(21) Appl. No.: 17/734,027

(22) Filed: Apr. 30, 2022

(51) Int. Cl. G16H 20/60

(52) U.S. Cl. CPC G16H 20/60

(57) ABSTRACT

A system may include one or more sensors which analyze a sample of human milk to be fed to a specific infant. The system may further include an artificial intelligence server including one or more processors implementing several artificial intelligence processes. The artificial intelligence server may receive sensor data generated by one or more sensors and analyze the sensor data to identify constituent elements of macronutrients and micronutrients in the sample of milk. The artificial intelligence server may further compare the constituent elements of macronutrients and or micronutrients in the sample of milk with nutritional guidelines and with nutritional protocols obtain from historical clinical data from infants with a clinical profile similar to that of the specific infant. The artificial intelligence server may further identify one or more disease risk scores for the specific infant. Methods implemented by the system are also disclosed.

### BACKGROUND

### 1. Technical Field

This disclosure relates generally to a system that identifies a personalized fortification of human milk to feed to infants, particularly low-birth-weight and preterm infants. The system further customizes a regularly scheduled feeding fortification of human milk for infants to optimize nutrition and growth according to an expected growth schedule and according to a particular infant's nutritional needs. The system may monitor growth of an infant and further identify risk scores for the onset of diseases based on the infant's clinical data. This disclosure further relates to methods which identify nutritional needs in infants, particularly preterm infants, customizes a nutritional protocol according to a particular infant's needs, and monitors growth and disease risk scores.

### 2. Description of the Related Art

Preterm infants, or low-birth-weight infants (e.g., those infants born weighing less than 5 pounds, 8 ounces or 2.5 kilograms) start life under very difficult circumstances. While mortality rates for infants born in much of the world are as low as they have ever been in any civilization, preterm birth rates have risen over the last several years and many preterm infants do not survive as they are not ready to survive on their own. Preterm infants are infants who are born before 37 weeks of gestation, according to many standards, including the World Health Organization, although the line between preterm and full term can be subjective. Suffice it to say, many of these infants would not survive if left to standard post-natal care. Indeed, many of these would likely not survive without spending time in a neonatal intensive care unit (NICU) where they are targeted with tailored treatments seeking to improve their fragile health by constant monitoring applications of highly sophisticated medical procedures where optimal nutrition is key for thriving.

Preterm infant care has improved significantly in the last several decades. Many preemies born today survive due to these improvements in care even though even just decades ago, they would not have survived. However, preterm infant care today could still be improved, particularly in determining optimal and personalized nutrition needs and disease risk scores that if high can trigger the appropriate feeding intervention for preterm infants. While nutritional needs of human beings are not totally understood, nutritional needs of infants are likely less understood. Attempts have been made to determine guidelines for feeding preterm babies. In the United States, guidelines for infant nutrition have been developed by the American Academy of Pediatrics (“AAP”) which are feeding guidelines used for infants, especially preterm infants. In Europe, the infant feeding guidelines are promulgated by The European Society for Paediatric Gastroenterology, Hepatology, and Nutrition (“ESPGHAN”). Other institutions have developed their own nutritional guidelines as a result of internal practice observations. While there are differences in these guidelines, essentially they consist of recommendations, for doctors who care for infants and preterm infants, that include the feeding frequency, the volume to be feed, and the macronutrients and micronutrient needed given the infants weight. These guidelines are specific to the infant's weight, but fail to address nutritional needs an infant may have due to the infant's clinical data, which may include a high predisposition for a certain diseases, in that infants who are likely to develop a particular disease may require more macro or micronutrient intake than another infant of the same weight not predisposed to that disease.

There is a general consensus amongst neonatologists that human milk is the best source of nutrition for any infant, not only because the nutrients in breastmilk are better absorbed and used by the infant, but also because human milk contains immunity-boosting elements (human milk oligosaccharides). However, human milk is highly variable in nutrient content and often does not address the nutritional needs of preterm infants, so in order to meet the requirements of nutritional guidelines the milk needs to be fortified. The objective of fortification is to increase the concentrations of both macro and micronutrients to levels such that at the suggested feeding volumes infants receive amounts of all nutrients that meet the chosen nutritional guidelines' requirements. Therefore in order for the milk to be fortified to the guidelines' recommended levels the milk needs to first be analysed for its macro and micronutrient content. Furthermore, while these nutritional guidelines are helpful to doctors in developing a feeding protocol for a preterm infant, they require a certain amount of monitoring, waiting, and checking to ascertain whether or not the protocol is adequate. In this short amount of time, some preterm infants may not survive due to a host of reasons, not the least of which is consuming too few grams of a macronutrient or missing fortifications that would, if correctly administered, prevent growth faltering (a growth rate below that which is appropriate for the infant) and life-threatening disease.

Therefore, there is a deep-felt need for a system which monitors a particular preterm infant's growth and determines an optimal and personalized nutritional protocol based on the analysis of the human milk for its macro and micronutrients, chosen nutritional guidelines, and on historical nutritional protocols obtained from large amounts of clinical data from infants with a similar clinical profile. The nutritional protocol would include the exact amount of macronutrients and micronutrient the infant requires, the fortification needed given the milk analysis, the fortifiers used, the feeding frequency, and the volume to be fed. In other words, there is a deep-felt need for personalized targeted fortification based on testing human milk for its macro and micronutrient components and fortifying the milk based on the needs of a specific infant. There is also an unmet need to provide a timestamp, blockchain identifier, or other indication of what personalized targeted fortification was given to an infant, which may include the infant's clinical profile, the infant's growth measurements (including weight, head circumference, and or length), the chemical analysis of the human milk used in the feeding, the nutritional guidelines used, the milk fortification performed for each macro or micronutrient, the fortifiers used, the volume actually feed to the infant, and the immediate feedback from the feeding (including the nutrition prescribed by the fortification versus the nutrition administrated). There is also a need to collect and analyse large quantities of clinical data from the parents and the infant, before birth, at birth, and after birth in order to accurately identify risk factors such as growth faltering and other diseases which may be affected by nutrition, and suggest the optimal milk fortification that can lower any high risk score at an early stage.

### SUMMARY

Disclosed herein is a system that may include one or more sensors which analyze the macronutrient and or micronutrient composition of a sample of human milk. The system may further include an artificial intelligence server including one or more processors implementing several artificial intelligence processes. The artificial intelligence server may receive sample data, generated by the sensor or sensors, and analyze the sensor data to identify constituent elements of macronutrients and micronutrients in a sample of milk. The artificial intelligence server may further compare the constituent elements of macronutrients and or micronutrients in the sample of milk with nutritional guidelines and provide nutritional recommendations for the fortification of the milk. The artificial intelligence server may further identify clinical data specific to a particular infant and identify disease risk scores for diseases or conditions which may be affected by nutrition, including but not limited to growth faltering (“GF”), bronchopulmonary dysplasia (“BPD”), necrotizing enterocolitis (NEC) and sepsis from historical clinical data obtained from one or more infants. The artificial intelligence server may further identify nutritional protocols based on historical data obtained from infants with a similar clinical profile as the infant. The artificial intelligence server may further compare the nutritional protocols based on historical data with the nutritional protocols based on nutritional guidelines, and identify the optimal personalized nutritional protocol to be fed to the specific infant. The artificial intelligence server may further compare the constituent elements of macronutrients and or micronutrients in the sample of milk scanned by the sensor with the optimal personalized nutritional protocol and provide recommendations for the fortification of the milk, including what fortifiers to use.

Further disclosed herein is a method which may include receiving, by an artificial intelligence server which includes one or more processors, sensor data generated by a sensor or sensors. The method may also include analyzing, by the artificial intelligence server which includes one or more processors, the sensor data to measure at least the concentrations of macronutrients and or micronutrients in the sample of milk, the milk freshness, and potentially the presence of metabolites of opioids, cannabinoids, or any other drug deemed appropriate. The method may further include comparing, by the artificial intelligence server which includes one or more processors, the concentrations of macronutrients and or micronutrients in the sample of milk for a particular infant with the suggested ranges provided by the chosen nutritional guidelines. If any macronutrient or micronutrient is less than the lower value of the range, the artificial intelligence server which includes one or more processors, may recommend a milk fortification to ensure that the final concentration of these nutrients are within the suggested ranges of the chosen nutritional guidelines. The method may also include identifying, by the artificial intelligence server which includes one or more processors, clinical data specific to a particular infant. The method may further include providing, by the artificial intelligence server which includes one or more processors, optimized nutritional recommendations for milk fortification to a device for presentation to a user.

### BRIEF DESCRIPTION OF THE DRAWINGS

The accompanying drawings illustrate various embodiments of the systems and methods disclosed herein.

FIG. 1 illustrates a method for determining an optimal personalized milk fortification for an infant.

FIG. 2 illustrates an exemplary system for determining an optimal nutrition recommendation for an infant.

FIG. 3 illustrates an exemplary sensor system for analyzing a milk sample.

FIG. 4 illustrates an exemplary optimal milk fortification scheme for a particular infant broken down by macronutrient.

FIG. 5 illustrates a block diagram of a artificial intelligence server.

FIG. 6 illustrates a method for identifying a milk fortification level for an infant based on nutritional guidelines.

FIG. 7 illustrates a method for identifying a milk fortification level for an infant exhibiting a high risk score for diseases or conditions which may be affected by nutrition, including but not limited to GF, BPD, NEC, and sepsis.

FIG. 8 illustrates a method for obtaining a recommended optimal and personalized milk fortification based on past subjects with similar clinical profile.

FIG. 9 illustrates a method of adding or attaching a feeding protocol for an infant to a blockchain.

FIG. 10 illustrates an artificial intelligence method for obtaining risk scores for diseases or conditions which may be affected by nutrition, including but not limited to GF, BPD, NEC, and sepsis.

### DETAILED DESCRIPTION OF PREFERRED EMBODIMENTS

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

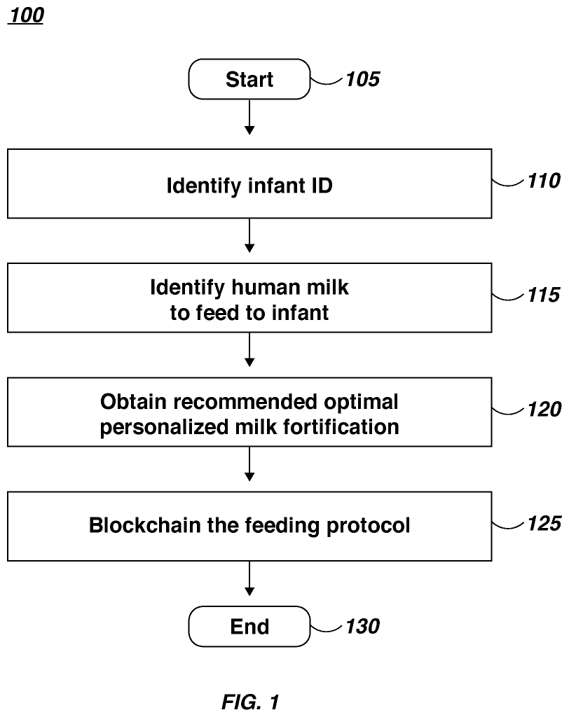

FIG. 1 illustrates a method 100 for determining an optimal personalized milk fortification for a baby. An “infant” or “baby” as used herein means a child who drinks human milk or receives parenteral nutrition as a primary source of nutrition. Human milk includes mother's milk, donor human milk, human milk based formula, and pools of donor milk. Infant children, babies, newborns, or children less than 1000 days old, and preterm newborns are collectively referred to as “infants” for the purposes of this disclosure.

Method 100 is an exemplary method that combines many elements of systems and methods herein as an overarching, but less detailed, example of the disclosure. Method 100 begins at start 105 with identifying an infant identification (“ID”) which may be a scanned or typed number, a QR code, a bar code, RFID, or any other way of uniquely identifying an infant known to those of ordinary skill in the art. Method 100 continues at step 115 where human milk is identified that is to be fed to an infant. The human milk may be provided by an infant's mother, milk donations to a milk pool, human milk based formula, or any other source of milk available to hospitals. Based on the human milk available to the infant, a recommended optimal personalized milk fortification may be obtained at step 120 using techniques that will be described below. At step 125, a feeding protocol may be attached to or inserted into a blockchain such that information about the feeding (e.g., amount eaten, amount regurgitated, fortifications provided, milk source, etc.) may be permanently stored in an unchangeable and authenticated format. Method 100 ends at step 130.

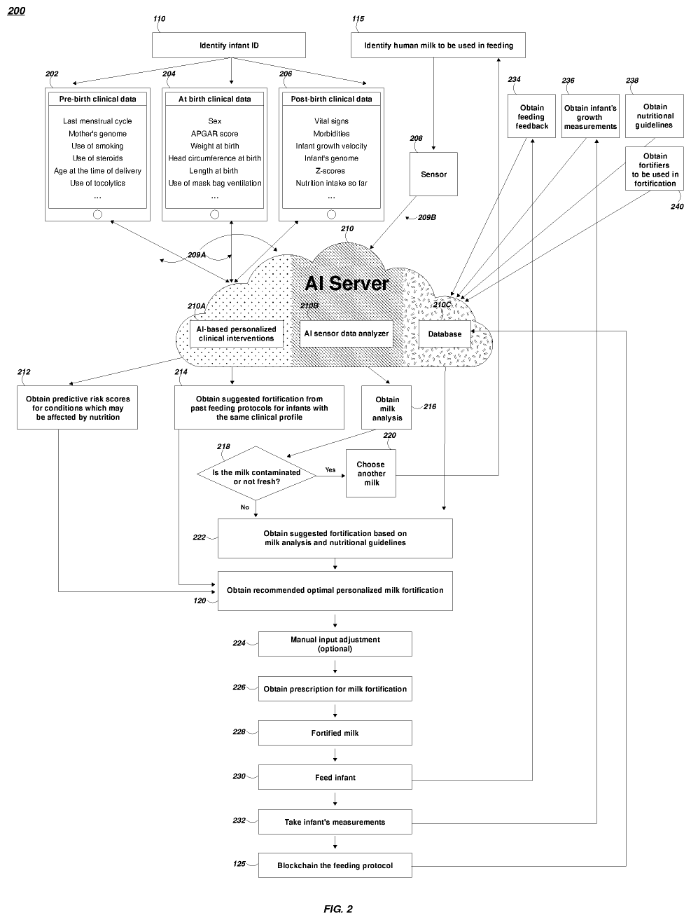

FIG. 2 illustrates an exemplary system 200 for determining an optimal nutrition recommendation for an infant. An infant ID identified in step 110 of system 100 may be provided to a device 202, a device 204, or a device 206, where these devices may or may not be distinct devices. Devices 202, 204, and 206, which may or may not be distinct devices, may be used by different users to provide information about an infant. For example, device 202 may be associated with a family member of an infant, such as a mother, and may collect data from the family member about the infant. Information collected on device 202 may be any pre-birth clinical data about the infant. Device 204 may collect information provided by hospital staff, such as nurses, nursing assistants, technicians, laboratory workers, and any other hospital staff. Information collected via device 204 may be clinical data about the infant at and around the time of birth. Device 206 may be associated with a doctor, a doctor's assistant, or a nurse, for example, and may collect data about the infant post-birth as post-birth clinical data. Devices 202, 204, and 206, which may or may not be distinct devices, may be a computing device that includes a processor. Examples of computing devices include desktop computers, laptop computers, tablets, smart phones, game consoles, personal computers, notebook computers, and any other electronic computing device with access to processing power sufficient to interact with system 200. Devices 202, 204, and 206, which may or may not be distinct devices, may include software and hardware modules, sequences of instructions, routines, data structures, display interfaces, and other types of structures that execute computer operations. Further, hardware components may include a combination of Central Processing Units (“CPUs”), buses, volatile and non-volatile memory devices, storage units, non-transitory computer-readable storage media, data processors, processing devices, control devices transmitters, receivers, antennas, transceivers, input devices, output devices, network interface devices, and other types of components that are apparent to those skilled in the art. These hardware components within devices 202, 204, and 206, which may or may not be distinct devices, may be used to execute the various methods or algorithms disclosed herein independent of or in coordination with other devices disclosed herein.

Each of devices 202, 204, and 206, which may or may not be distinct devices, may be used to collect different clinical data about a particular infant. The clinical data may include information such as birthweight of a baby taken just after birth, gestational age of a baby which is taken from the beginning of the mother's last menstrual period, sex or lack of sex of the baby (e.g., androgenous babies), APGAR score which is indicative of how well the baby is doing after birth, multiple birth which includes information about whether or not the pregnancy included two or more fetuses; the use of prenatal steroids to support lung development in a fetus; the existence of maternal pregnancy-induced hypertension, baby blood pressure, mother's blood pressure; baby blood pH, mother's blood pH, the age of the mother at the time of delivery, head circumference of the baby, length of the baby, parity which is indicative of the number of times a mother has given birth to a fetus with a gestational age of 24 weeks or more, white blood cell count; baby body temperature, mother's body temperature, baby respiratory rate, mother's respiratory rate, race of mother and baby, mother's disorders, hematocrit which is indicative of a ratio of the volume of red blood cells to the total volume of blood for mother and or baby, heart rate of mother and or baby, sodium levels in mother and or baby, respiratory distress syndrome in a baby, prenatal care provided to a mother during pregnancy, partial pressure of oxygen as an amount of oxygen gas dissolved in the blood of mother and or baby, platelet count of mother and or baby, placental/uterine disorders during pregnancy, glucose levels In mother and or baby, sex of a baby, fraction of inspired oxygen as an estimation of the oxygen content in a mother and or baby, whether or not a delivery hemorrhage occurred during delivery; chromosome or congenital anomalies, chorioamnionitis which is indicative of inflammation of fetal membranes usually due to bacterial infection, tocolytics which are medicines used to suppress premature labor, oxygen saturation percentages of mother and or baby, the need for respiratory support of mother during labor and or baby post birth, premature rupture of membrane which is the tearing of membranes of the amniotic sac before labor begins, the existence of labor and delivery complications, a partial pressure of carbon dioxide in mother and or baby, an oxygenation index which is indicative of the efficiency of oxygen exchange in the lungs, a neutrophil count which may be indicative of an infection, inflammation, leukemia, and other conditions; a level of maternal education; whether or not the mother has developed gestational diabetes or diabetes mellitus during pregnancy; whether the baby was born inside a tertiary center or outside of a tertiary center, a delivery mode (e.g., natural unassisted childbirth, assisted childbirth, or cesarian section), the existence of an umbilical cord disorder, whether or not the baby was subjected to mask bag ventilation upon delivery, whether or not antibiotics have been administered to the mother and or baby, age of the baby post-delivery, urine output from the baby over a selected period of time, an indication of whether or not the baby is small for gestational age, whether or not the baby experiences seizures, oligohydramnios which refers to a volume of amniotic fluid that is less than expected for gestational age of a baby, mother's marital status, whether or not delivery of a baby occurred at a hospital, whether or not a physician was involved in the delivery, a death rate in a particular facility for infants in similar conditions, birth date of the baby, and baby apnea.

Other clinical data may include genetic, microbiome, and or epigenetic information. Genetic information may indicate that an infant is predisposed to a particular disease or ailment based on the infant's DNA or the infant's parents' DNA. Similarly, information about the infant's microbiome may be identified as clinical data by which disease predispositions may be measured. Microbiome data used as clinical data may identify a type of bacteria or a relative amount of one type of bacteria as compared with another bacteria (a healthy balance of bacteria) in the infant's microbiome and percentage thresholds for certain types of bacteria. Epigenetic information may indicate potential environmental triggers of genetic predispositions of a child. Other clinical data may take into account a change in an infant's growth over a period of time (A) as a relative percentile as compared with other infants. Another type of clinical data may include a mother's or donor's milk, whose composition changes over time, and whether they may or may not meet the nutritional needs of the infant.

Clinical data may include high and low thresholds, as needed, to specify tolerances for acceptable variation for this clinical data as a way to provide a risk score for early diagnosis and early treatment. These examples of clinical data are merely illustrative and do not constitute a complete list of all possible clinical data that may be used to determine an optimal nutrition or milk fortification recipe. Other clinical data known to those of skill in the art may also be considered to identify disease- or ailment-based risks or risks associated with malnutrition. Clinical data from devices 202, 204, and 206, which may or may not be distinct devices, may be provided to AI server 210 (shown in FIG. 2 as AI-platform), as will be discussed below.

Once an infant is born, step 115 of method 100 shown in FIG. 1 includes identifying human milk to be used in feeding the infant. To accomplish step 115 of method 100, a sensor 208 and an AI sensor analyzer 2010B are provided in system 200 which may test and identify characteristics of human milk for feeding the baby. Sensor 208 may be a spectrometer. Sensor 208 may be implemented with a near infrared sensor and emitter. Other types of sensors and emitters that are based on infrared, mid-infrared, gamma rays, X-rays, Raman spectrometers, or other wavelengths of light or detectors may also be used within sensor 208 alone or in any combination. Sensor 208 may also be an electrochemical sensor to collect data from a milk sample. Data collected by sensor 208 may be representative of a spectrographic sample and include data representative of the constituent material of the sample in either atomic or molecular form. In particular, sensor 208, via AI sensor data analyzer 210B, may obtain data representative of constituent concentrations of milk, which include carbohydrates (lactose and HMOs), fats, protein, vitamins, minerals, and any other component of milk. Carbohydrates, fats, and protein may be referred to as macronutrients while vitamins and minerals may be referred to as micronutrients. AI sensor analyzer 210B may provide information in percentage terms or in terms of macronutrients and or micronutrients per gram of milk or in any useful term of expressing the composition of a milk sample, including in metadata, text data, computer program data, using any known or convenient data format, and may include wireless delivery of data representative of the milk sample collected from sensor 208 and AI sensor data analyzer 210B.

In some embodiments, sensor or sensors 208 may provide this data by wired or wireless connection 209B to AI server 210. Data may also be provided manually from the sensor to AI server 210. Likewise, devices 202, 204, and 206, which may or may not be distinct devices, may provide clinical data to AI server 210 via a wired or wireless connection 209A.

AI server 210 may be implemented as one or more server computing devices. AI server 210 may include cloud computers, supercomputers, mainframe computers, application servers, catalog servers, communications servers, computing servers, database servers, file servers, game servers, home servers, proxy servers, stand-alone servers, web servers, combinations of one or more of the foregoing examples, and any other computing device that may be used to execute or perform machine learning, produce and run an artificial intelligence server within AI server 210, and implement a visual representation of the stored data. The one or more server computing devices may include software and hardware modules, sequences of instructions, routines, data structures, display interfaces, and other types of structures that execute server computer operations. Further, hardware components may include a combination of Central Processing Units (“CPUs”), buses, volatile and non-volatile memory devices, storage units, non-transitory computer-readable storage media, data processors, processing devices, control device transmitters, receivers, antennas, transceivers, input devices, output devices, network interface devices, and other types of components that are apparent to those skilled in the art. These hardware components within one or more server computing devices may be used to execute the various methods or algorithms disclosed herein, and interface with devices 202, 204, and 206, which may or may not be distinct devices, and sensor 208. AI server 210 may include various subcomponents implemented by the foregoing hardware, such as an artificial intelligence interface 210A which provides artificial intelligence based personalized clinical interventions for the identified infant. AI server 210 may further include a subcomponent 210B which analyzes sensor data from sensor 208. AI server 210 may also include a database subcomponent 210C that may act as volatile or non-volatile memory for executing artificial intelligence routines and successive artificial intelligence model testing.

Artificial intelligence interface 210A may provide predictive risk scores 212 for diseases and conditions which may be affected by nutrition, including but not limited to growth faltering (“GF”), bronchopulmonary dysplasia (“BPD”), necrotizing enterocolitis (“NEC”), and sepsis. Artificial intelligence interface 214 may also obtain suggested fortifications from past feeding protocols to or for infants with the same or similar clinical profiles as determined based on an analysis by AI server 210 of clinical data received for infants with different infant IDs. In this manner, AI server 210 may obtain a recommended optimal personalized milk fortification discussed above with respect to step 120 of method 100 in FIG. 1. The recommended optimal personalized milk fortification may be based on the AAP, the ESPGHAN, or particular NICU guidelines but may be specific to both the milk provided by a mother (or milk provided through a milk bank or human-milk formula) and to the baby's particular needs based on any of the foregoing clinical data. For example, research shows a higher percentage of protein in the baby's nutrition may be more useful for male babies than for female babies, and a higher percentage of fat in the baby's nutrition may be more useful for female babies than for male babies while sex-unassigned (e.g., androgenous) babies may need a combination of both. In another example, if a baby shows a particular susceptibility to a certain disorder and a medication or nutrient may prevent or lessen the adverse effects of the disorder, AI server 210 may account for that issue and modify AAP or ESPGHAN or any other guideline and recommendations accordingly to ensure that the mother's milk is properly fortified to provide the baby with the nutrition necessary to maximize survivability and thriving of the baby.

AI server 210 may obtain, via AI sensor data analyzer 210B, an analysis 216 of milk tested by sensor 208. AI sensor data analyzer 210B may determine whether or not the milk is fresh or contaminated 218. If the AI sensor data analyzer 210B detects that the milk is not fresh or not contaminated (218—“Yes”), AI server 210 may indicate that the milk is contaminated to a user and identify another source of milk listed in database 210C or from other sources to identify human milk to be used in feeding, as discussed in step 115 of method 100. If the milk is not contaminated and is fresh (218—“No”), AI server 210 may obtain a suggested fortification based on milk analysis and nutritional guidelines stored in database 210C. The nutritional guidelines stored at step 236 may be based on the AAP, the ESPGHAN, or specific NICU guidelines for nutrition for infants. Database 210C may also include information 240 about fortifiers at step 240 which are available to be administered in fortified milk

AI server 210 may, at this point, obtain a recommended optimal personalized milk fortification 120 based on the suggested fortification of the milk 222, the predictive risk scores 212, and the suggested fortifications from past feedings protocols to or for infants with the same or similar clinical profiles 214. Additionally, based on known predispositions or clinical data, AI server 210 may identify that an infant's nutritional intake should be fortified with an increased amount of certain macronutrients and or micronutrients to lower the risk of an infant contracting the disorder.

In some cases, a doctor may interact with, for example, device 206 and AI server 210 to optionally manually adjust the recommended optimal personalized milk fortification and write a prescription for milk fortification 224. Based on recommendations from the AI server 210, and approval or modification by a prescribing physician, a fortified milk prescription 226 may be generated.

In response, a fortified milk 228 may be produced to feed to infant at step 230. Once the baby has been fed, device 206 may be used by the physician or the hospital staff to report or provide information on each baby feeding. For example, feeding feedback information may include information about the feeding session, including amount consumed by an infant, amount regurgitated by the infant, duration of the feeding time, and any other relevant indicator known to those of ordinary skill of the art. Feeding feedback information may also include information about growth of the infant from day to day, including weight information, length information, head circumference, growth velocity, Z-scores, oxygen breathing volume information, heart rate information, urine and stool output information, and any other clinical data described herein or known to one of ordinary skill in the art, to determine an amount of the prescribed and fortified milk that was actually ingested by the baby may be obtained during or after feeding 230 at step 234. Measurements of the infant may be taken at step 232, from which growth measurements may be obtained at step 236, and the feeding protocol information may be inserted into or added to a blockchain at step 125 and or stored in database 210C.

Based on this feedback information provided via device 206 to AI server 210, future feedings may be adjusted to either increase or decrease the amount of donor milk fortified prescribed and the constituent solution for macronutrients, micronutrients, and fortifications may be adjusted based on how much milk the baby actually consumes to ensure that the baby is meeting survivability and growth thresholds day to day. Further, based on information provided to AI server 210, AI server 210 may modify or update the suggested milk fortification recipe. As a baby grows, for example, different milk fortification recipes may be suggested. As the baby increases in weight, the fat content, for example, may be decreased while the protein content is increased. If an indicator of a disease is apparent in clinical data obtained for the infant, fortifications may be suggested by AI server 210. For example, if an ailment or a disease, such as bronchopulmonary dysplasia, sepsis, necrotizing enterocolitis, or another morbidity, is present in an infant, AI server 210 may adjust a recommended fortification based on the level of the risk score for that disease. AI server 210 may also include information about the donor milk available for fortification. For example, if an infant is born without the ability to digest fat, AI server 210 may suggest a particular donor milk or particular donor milk pool with the lowest possible fat to feed a particular baby for optimal fortification of milk in this and similar situations.

AI server 210 may use the foregoing clinical data, and or feedback from the results of care of other similarly situated infants, and feedback from the care of a particular infant to derive a risk score for the infant based on a collection of variables representative of the clinical data and feedback in view of or not in view of growth charts used by a particular facility. In addition, AI server 210 may further provide suggestions for the nutritional or feeding protocol based on past interventions for infants with similar clinical profiles, regardless of hospital or location, to provide a data-driven evidence-based course of treatment for neonatologists or other healthcare professionals. AI server 210 may iteratively learn on a recommendation-by-recommendation basis and an outcome-by-outcome basis how to successfully treat infants with similar conditions and provide that information to care providers in any facility to ensure that the type feeding protocol and adequate feeding protocol is available for an infant with certain risk factors, clinical data, or needs.

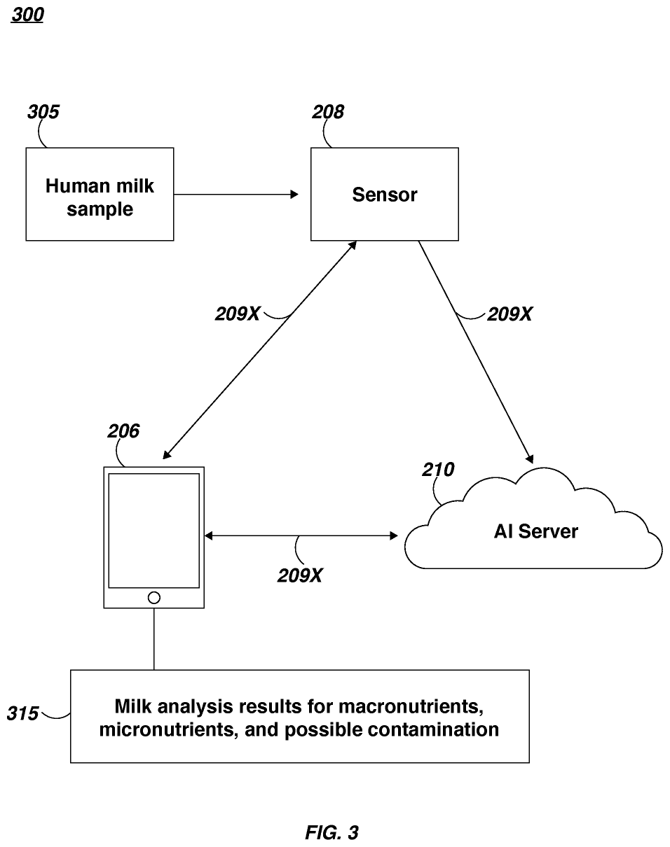

FIG. 3 illustrates an exemplary sensor system 300 for analyzing a milk sample 305. System 300 may include a sensor or sensors 208 which in conjunction with AI sensor data analyser 210B, residing inside AI server 210, analyzes a sample 305, such as a sample of mother's milk or donor's milk. It should be noted that infants who receive exclusively parenteral nutrition need not use sensor 208 as they do not receive human milk as nutrition although system 300 may still track the nutrition provided to the infant and track the infant's growth. Sensor 208 may be a spectrometer. Sensor 208 may be implemented with a near infrared sensor and emitter. Other types of sensors and emitters that are based on infrared, mid-infrared, gamma rays, X-rays, Raman spectrometers, or other wavelengths of light or detectors may also be used within sensor 208 alone or in any combination. Sensor 208 may also be an electrochemical sensor or a colorimetric method providing either quantitative or semiquantitative data from sample 305. Data collected by sensor 208 may be representative of a spectrographic sample and include data representative of the constituent material of the sample in either atomic, molecular, or ionic form. In particular, sensor 210 may obtain data representative of constituent components of milk, which include carbohydrates (consisting of lactose and human milk oligosaccharides), fat, protein, vitamins, minerals, and any other component of milk. Carbohydrates (consisting of lactose and human milk oligosaccharides), fat, and protein may be referred to as macronutrients while vitamins and minerals may be referred to as micronutrients. Sensor 208 may provide information in percentage terms or in terms of macronutrients and micronutrients per gram of milk or in any useful term of expressing the composition of sample 305, including in metadata, text data, computer program data, using any known or convenient file format, and may include wireless delivery of data representative of sample 305 collected from sensor 208. The data may include milk analysis 315 results for macronutrients, micronutrients, and possible contamination of the milk (opioids, cannabinoids or other contaminates), as well as levels of milk freshness.

In some embodiments, sensor 208 may provide this data by wired or wireless connections 212X to AI server 210 or to a device 206, such as a tablet. Data may also be provided manually from the sensor 208 to AI server 210 or device 206. Sensor 208 may communicate any data generated by sensor 208 directly to device 206 by communication link 209X or to AI server 210 by communication link 209X. Further, AI server 210 may exchange data with (e.g., send and receive data from) device 206 through communication link 209X. Communication link 209X may be facilitated with any known method of communication. For example, communication links may each use wired or wireless communication using any desired protocol to communicate information between sensor 208, AI server 210, and device 206.

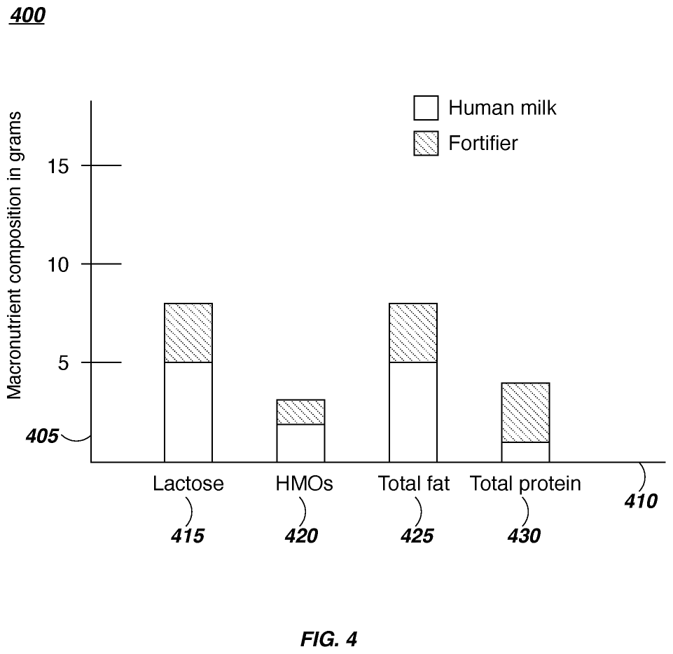

FIG. 4 illustrates an exemplary optimal milk fortification scheme 400 for a particular infant. Milk fortification scheme 400 may show a particular recipe, suggestion, or recommendation for milk fortification for a particular infant. As shown in scheme 400, a bar graph includes a vertical axis 405 which identifies a macronutrient composition in grams per 100 grams of a particular milk before and after fortification and a horizontal axis 410 which includes the macronutrients of the human milk before fortification as well as after fortification. For example, macronutrients such as lactose 415, human milk oligosaccharides (“HMOs”) 420 (which together with lactose 415 form carbohydrates), a total fat 425, and a total protein 430 are provided along the horizontal axis 410. Each of the bars representative of lactose 415, HMOs 420, total fat 425, and total protein 430 comprise a representation of a portion of human milk included within each of the bars and a representation of an amount of fortifiers in grams added to grams of the human milk which may include macronutrients and or micronutrients necessary for optimal nutrition for a particular infant. The particular unfortified milk shown, for purposes of explanation only, may be displayed, for example, on device 206, and includes a recommendation for fortifying milk that includes 5 grams of lactose 415, 2 grams of HMOs, 5 grams of total fat, and 1 gram of total protein with fortifications of 3 grams of lactose, 1 gram of HMOs, 3 grams of total fat, and 3 grams of total protein which are needed to achieve optimal nutrition for a particular infant. It should be noted that axis 405 can be shown in grams or in relative percentages of constituent elements of a milk solution when displayed on device 206.

As previously discussed, AI server 210, shown in FIG. 2, may use an AI sensor data analyzer 210B and artificial intelligence models to develop an optimal fortification scheme for a particular milk sample. For example, the amount of fat, carbohydrates, protein, and fortifiers in a particular milk sample may be determined by AI server 210 and provided to device 206, for example, to provide suggestions, such as a visual indicator or a text-based list of ingredients, macronutrients and or micronutrients, or other fortifications to add to milk for optimal nutrition of an infant.

In this way, optimal nutrition for an infant may be iteratively and successively improved by the use of artificial intelligence to not only identify optimal nutritional protocol but also provide likelihoods of success/mortality for a particular infant and risk scores for diseases that can be affected by nutrition including but not limited to GF, BPD, NEC, and sepsis. The use of artificial intelligence may drastically improve the odds that a particular infant will survive based on receiving optimal nutrition in the milk that the infant ingests as well as milk fortification prescriptions that have helped, in the past, infants in similar conditions thrive. These iterative and successive improvement techniques will be described in more detail below.

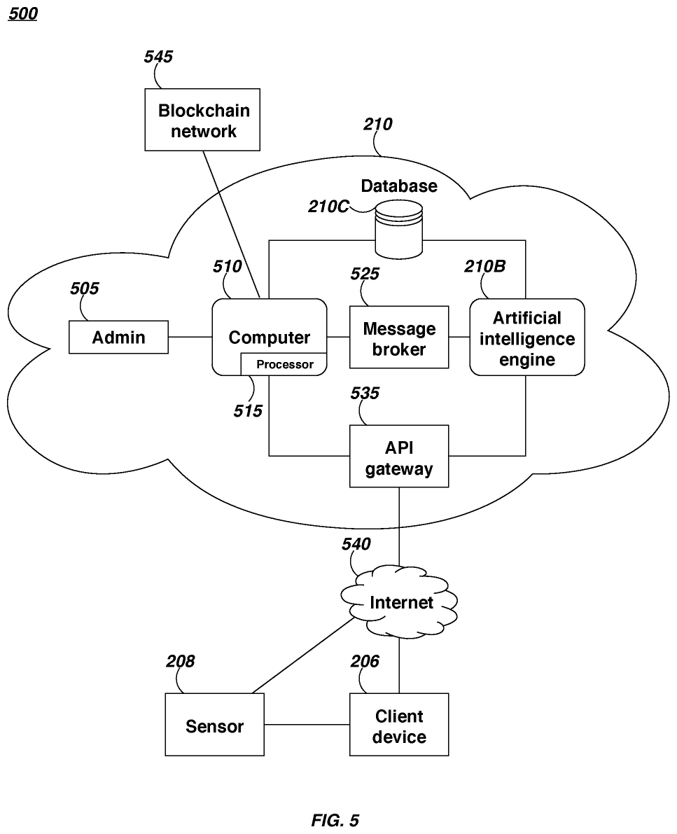

FIG. 5 illustrates a block diagram of an AI server 210. AI server 210 may receive information from the sensor or sensors 208 or the client device 206 through the internet 540. Data and server information can also be retrieved from the AI server 210 by the client device through the same internet 540 connection. As indicated by the FIG. 5, all the communications go through an API gateway 535 that works as a firewall that protects the AI server 210 from outside attacks and also forwards the incoming messages to their correct destinations.

As shown in FIG. 5 AI server 210 may include an admin backend access point 505 where administrators may monitor AI server 210 and inspect data produced by AI server 210 as well from the client device 206 and sensor 208. Admin backend access point 505 may also be used to analyse the system performance as well to correct possible issues. Database 210C contains all the information in an encrypted format and only readable with the unlocking user key, the database 210C may also have daily backup to avoid any loss of data. AI server 210 may further include a server computer processor 515 which processes data received from sensor 208 along with data from client device 206, database 210C and information created by artificial intelligence engine 210B. Artificial intelligence engine 210B may include a processor and apply artificial intelligence models that apply machine learning algorithms to sets of data from database 210C and sensor 208 to identify risk scores for an infant developing a disease or an ailment, for example, or may identify risk scores for malnutrition in infants, especially preterm infants. A message broker 525 may be implemented to transform and route messages between server computer processor 515 and artificial intelligence engine 210B to mediate the communications. Device 206 may be operated by a doctor, hospital staff, or a family member (as discussed with respect to devices 202, 204, and 206 in FIG. 2). An internal connection may be created from the computer processor 515 to an existing blockchain network to store permanently the feeding protocol, this information will be encrypted and cannot be tracked back to any private user information.

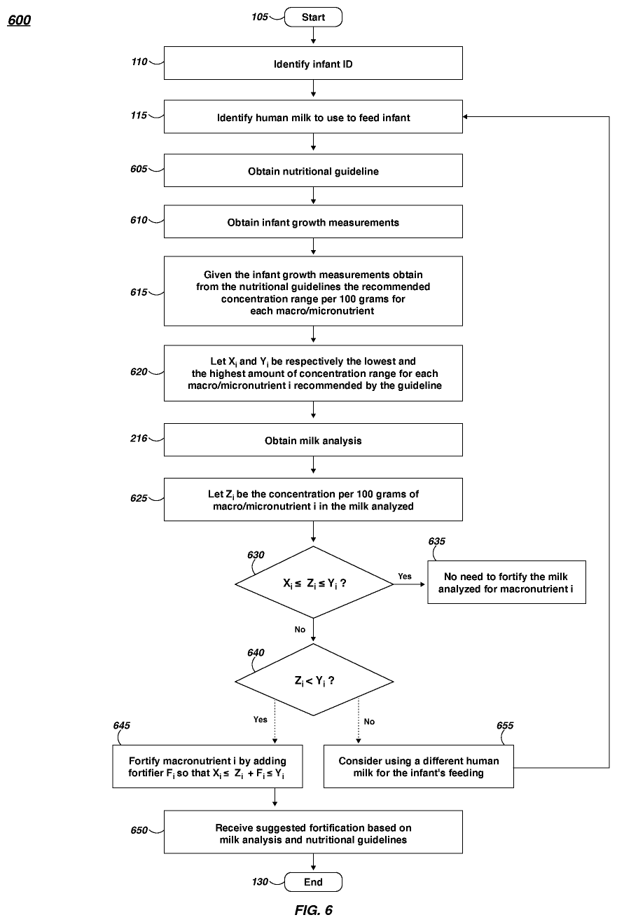

FIG. 6 illustrates a method 600 for identifying a milk fortification level for an infant based on the milk analysis and the chosen nutritional guideline. Method 600 incorporates elements of method 100, shown in FIG. 1, and method 200 while explaining details of the various steps in method 100 and in method 200. Method 600 starts at step 105 and identifies an infant ID at step 110. At step 115, method 600 identifies human milk to use to feed an infant whether the milk is mother's milk, milk from a donor, milk from a donor pool, or human milk formula. At step 605, method 600 may invoke step 238 using a processor or processors, such as processor 515 in AI server 210 to obtain nutritional guidelines, such as nutritional guidelines for infants or premature infants promulgated by the AAP, ESPGHAN, or that are specific to a particular hospital or NICU (neonatal intensive care unit). At step 610, processor 515 may obtain infant growth measurements by invoking step 236 using a processor or processors, such as processor 515 in AI server 210, for example. At step 615, processor 515 may obtain from the nutritional guidelines a recommended concentration range per amount (e.g., 100 grams) for each macro or micronutrient given the infant growth measurements. At step 620, the processor may identify a range for each macro/micronutrient “i” recommended by the guideline where Xᵢ and Yᵢ are respectively the lowest and highest amounts the range specified by the nutritional guideline. Processor 515 may obtain a milk analysis from sensor 208 via AI sensor data analyzer 210B at step 216 to determine the macro or micronutrient concentrations in a particular sample of milk. At step 625, processor 515 may identify a concentration Zᵢ of a particular macro or micronutrient “i” in an amount (e.g., 100 grams) of the milk analyzed.

At step 630, using a processor or processors, such as processor 515 in AI server 210 may perform a comparison to determine whether Zᵢ is within the nutritional guideline range in that Zᵢ is higher than the minimum concentration Xᵢ and lower than the maximum concentration Yᵢ. If Zᵢ is in the range between Xᵢ and Yᵢ, (630—yes) processor 515 determines, at step 635, that there is no need to fortify the milk analyzed for macro or micronutrient “i.” However, if processor 515 determines that Zᵢ is outside the range (630—no), processor 515 determines at step 640 whether or not Zᵢ is less than Xᵢ. If Zᵢ is less than Xᵢ, (640—yes) processor 515 may indicate that a fortification of macro or micronutrient “i” is necessary such that the addition of fortifier Fᵢ ensures that Zᵢ is greater than the minimum recommendation for the guideline, Xᵢ and less than the maximum recommendation for the guideline Yᵢ. At step 650, processor 515 may transmit a suggested fortification based on the milk analysis and nutritional guidelines to a user device, such as user device 206, for review by a neonatologist or the healthcare professional caring for the infant.

If at step 640, Zᵢ is not less than and not equal to (greater than) Yᵢ (640—no), processor 515 may recommend that another source of milk be found for a particular infant at step 655 and return to step 115 to identify another source of milk from mother's milk, a milk pool, a milk donor, or human milk formula. It should be noted that too much of a particular concentration of a macro or micronutrient can predispose the infant to various conditions including diabetes, renal disorders and cardiovascular issues. Method 600 ends at step 130.

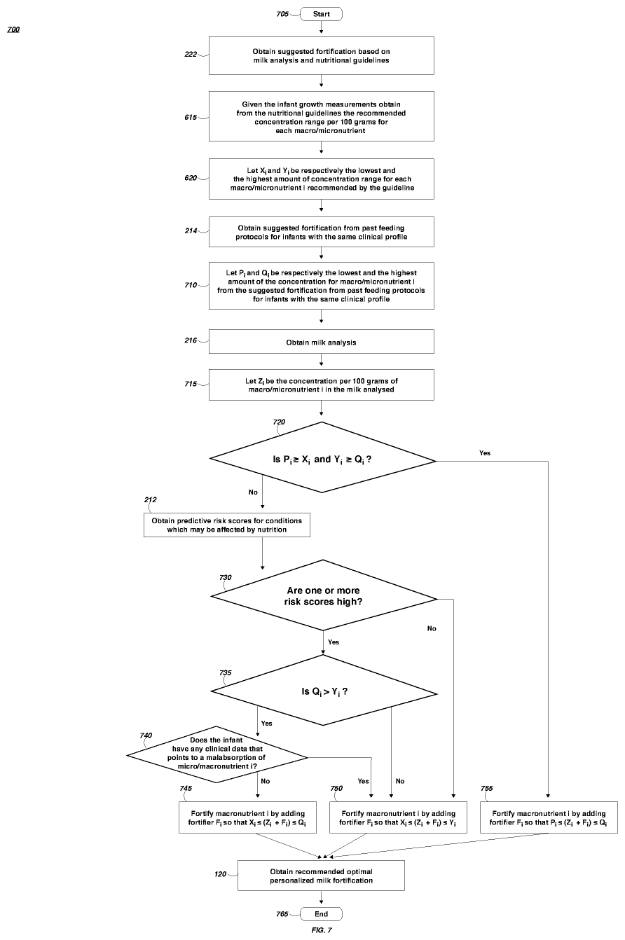

FIG. 7 illustrates a method 700 for identifying a milk fortification level for an infant with elements of method 600, based on the milk analysis and the chosen nutritional guideline, with historical milk fortifications given to infants with the same clinical profile. Method 700 identifies the optimal milk fortification by also taking into consideration the infant risks scores for diseases which can be affected by nutrition, including but not limited to Growth Faltering (GF), Bronchopulmonary Dysplasia (BPD), Necrotizing enterocolitis (NEC), and sepsis. Method 700 starts at step 705 where processor 515 is initiated to obtain a suggested fortification based on milk analysis and nutritional guidelines at step 222. At step 615, a processor or processors such as processor 515 may obtain from the nutritional guidelines a recommended concentration range per amount (e.g., 100 grams) for each macro and micronutrient given the infant growth measurements. At step 620, the processor may identify a range for each macro or micronutrient “i” recommended by the guideline where Xᵢ and Yᵢ are respectively the lowest and highest amounts of the range specified by the nutritional guideline. At step 214, processor 515 may obtain a suggested fortification from past feeding protocols to infants with the same or similar clinical profile based on clinical data collected via devices 202, 204, and 206, which may or may not be distinct devices. At step 710, processor 515 may identify a lowest and highest amount of concentration for the macro or micronutrient “i” from the suggested fortification from past feeding protocols for an infant with the same or similar clinical profile where the lowest concentration for macro or micronutrient “i” is designated as Pᵢ and a highest concentration for macro or micronutrient “i” is designated as Qᵢ. At step 216, processor 515 may obtain a milk analysis from sensor 208 via AI sensor data analyzer 210B for milk that is available to feed an infant. The milk may be mothers' milk, donor milk, milk from a donor pool, or human milk formula. At step 715, processor 515 may identify a concentration per amount (e.g., 100 grams) of macro or micronutrient “i” in the milk analyzed in step 216 and designate that identified concentration as Zᵢ.

At step 720, processor 515 may determine if Pᵢ is equal to or greater than Xᵢ and if Yᵢ is equal to or greater than Qᵢ. That is, processor 515 may determine if Xᵢ<Pᵢ and Qᵢ<Yᵢ, in other words the concentration range of macro or micronutrient “i” obtained from past feeding protocol to infants with the same or similar clinical profile is inside the range recommended by the chosen nutritional guidelines for the same macro or micronutrient “i”. If Xᵢ<Pᵢ and Qᵢ<Yᵢ (720—yes), processor 515 determines that macro or micronutrient “i” is to be fortified by adding fortifier Fᵢ such that Pᵢ is less than or equal to Zᵢ plus Fᵢ and which is also less than or equal to Qᵢ at step 755. In other words, in this case the suggested fortification the range should be the one obtained from past feeding protocol to infants with the same or similar clinical profile and not the range from the nutritional guidelines.

If Pᵢ is not equal or greater than Xᵢ or Yᵢ is not equal or greater than to Qᵢ, (720—no), processor 515 may obtain predictive risk scores for diseases which may be affected by nutrition, including but not limited to GF, BPD, NEC, and sepsis, at step 725. Processor 515 may determine at step 730 whether or not these predictive risk scores generated at step 725 are high at step 730. If the risk scores generated at step 725 are determined to be high (step 730—yes), processor 515 may determine whether or not Qᵢ is less than or equal to Yᵢ at step 735. If processor 515 determines that Qᵢ is greater than Yᵢ at step 735 (735—yes), this means that the suggested concentration range of macro or micronutrient “i” obtained from past feeding protocol is higher than the one suggested by the nutritional guidelines, so the historical data shows that higher concentrations of the macro or micronutrient “i” were successfully used to address high risk scores for certain diseases. In this case, the processor 515 may use clinical data to determine whether or not the infant has malabsorption issues with the specific micro/macronutrient “i”. If clinical data indicates that no malabsorption issue exists for macro or micronutrient “i” (740—no) at step 740, processor 515 may determine that fortifier Fᵢ when added to Zᵢ should be less than or equal to Qᵢ but greater than or equal to Xᵢ at step 745. If clinical data indicates that there is a malabsorption for the macro or micronutrient “i” (750—yes) then the ranged used for the milk fortification should be the one suggested by the nutritional guidelines and therefore processor 515 may determine that macro or micronutrient “i” should be added such that fortifier Fᵢ added to Zᵢ is less than Yᵢ but greater than Xᵢ at step 750. At this point, if processor 515 has determined that the risk scores are not too high (730—no), or that Qᵢ is less than or equal to Yᵢ (735—no), then the ranged used for the milk fortification should also be the one suggested by the nutritional guidelines and therefore processor 515 may determine that macro or micronutrient “i” should be added such that fortifier Fᵢ added to Zᵢ is less than Yᵢ but greater than Xᵢ at step 750.

Fortification levels determined at steps 745, 750, and 755 may be used by processor 515 to determine a recommended optimal personalized milk fortification level for a particular infant 120 based on other infants with similar clinical situations and a milk analysis for a particular milk sample. At step 765, method 700 ends.

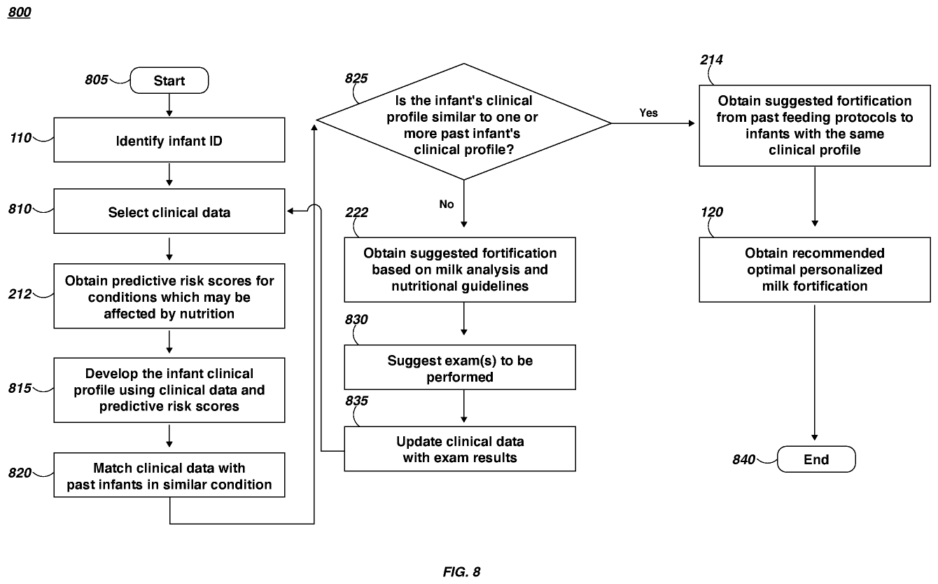

FIG. 8 illustrates a method 800 for obtaining a recommended optimal and personalized milk fortification obtained from past feeding protocol to infants with the same or similar clinical profile. Method 800 may be performed by, for example, processor 515 in AI server 210 discussed with respect to FIG. 2 and FIG. 5. Method start 805 may implement processor 515 to identify a particular infant ID at step 110. At step 810, processor 515 may identify or receive an indication of one or more elements of clinical data associated with the infant identified in step 110. At step 212, processor 515 may obtain predictive risk scores for diseases which may be affected by nutrition, including but not limited to GF, BPD, NEC, and sepsis. At step 815, processor 515 may create an infant clinical profile using collected clinical data and predictive risk scores. Based on the clinical profile developed in step 815, processor 515 may match clinical data with other infants who experienced similar conditions at step 820. At step 825, processor 515 may determine whether or not the infant's clinical profile is similar to one or more other infants who experienced similar conditions. If processor 515 does find one or more infants who experienced similar conditions in database 210C, for example, (825—yes), processor 515 may obtain a suggested fortification from past feeding protocols for infants with the same or similar clinical profile at step 214 and generate a recommended optimal personalized milk fortification at step 120.

If, however, no clinical profile for other infants is the same or similar to the infant (825—no), processor 515 may obtain a suggested fortification based on milk analysis and nutritional guidelines at step 222 and suggest exams to be performed at step 830. If there are no suggestions available for further exams then return the value from step 222. The results of those exams suggested in step 830, for example, may be used to update clinical data at step 835 which may be provided back to processor at step 810. A suggested fortification based on milk analysis and nutritional guidelines may be provided at step 222 and processor 515 may obtain a recommended optimize personalized milk fortification at step 120. Method 800 ends at step 840.

Over time, AI server 210 may track progress (e.g., a A positive/negative growth) of an infant based on the clinical data selected at step 810. The new optimal personalized milk fortification may be tested over time to track the progress of infant in terms of clinical data which may be further analyzed by method 800 beginning at step 810.

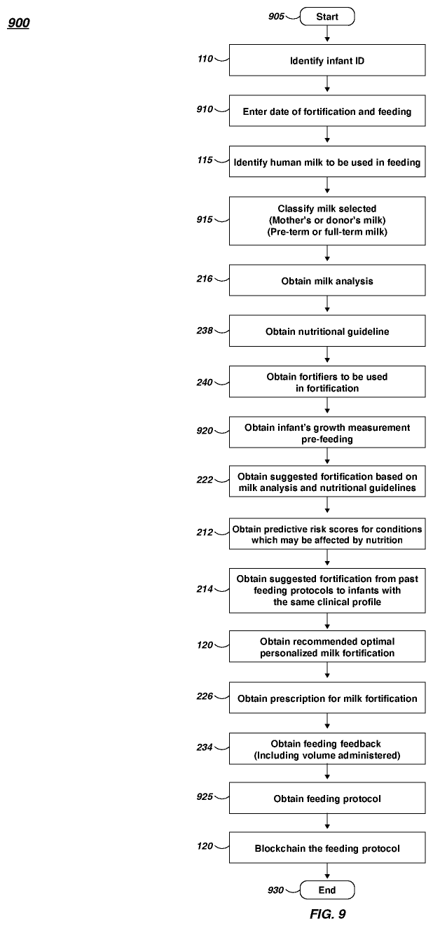

FIG. 9 illustrates a method 900 of adding or attaching a feeding protocol for an infant to a blockchain. Method 900 starts at step 905 where a processor or processors, such as processor 515, is initiated to identify an infant ID at step 110. At step 910, processor 515 may enter a date for the milk fortification and feeding into database 210C, for example. Processor 515 may also identify human milk to be used in feeding from any source, such as mother's milk, milk donors, milk donor pools, or human milk formula at step 115. Once the human milk is identified at step 115, processor 515 may classify the selected milk as being sourced from the infant's mother, a milk donor, or a milk donor pool, and whether the milk is preterm milk or full-term milk at step 915. The selected milk may be analyzed by sensor 208 via AI sensor data analyzer 210B at step 216 while processor 515 obtains nutritional guidelines for a particular infant at step 238. Processor 515 may query database 210C to determine which fortifiers are available and which fortifiers are to be used in fortification at step 240.

Processor 515 may further obtain growth measurement information about the infant before feeding at step 920. Processor 515 may further obtain a suggested fortification based on the milk analysis and nutritional guidelines at step 222. Processor 515 may further obtain a suggested fortification from past feeding protocols to infants with the same clinical profile step 214. Processor 515 may further generate predictive risk scores for diseases which may be affected by nutrition, including but not limited to GF, BPD, NEC, and sepsis at step 212. At step 120, processor 515 may generate a recommended optimal personalized milk fortification and provide the information to device 206, for example for review by a doctor and to obtain a prescription for the fortification at step 226. The infant may be fed the fortified milk and processor 515 may receive feeding feedback including the volume administered and the volume consumed at step 234 to obtain the feeding protocol at step 925. The feeding protocol may then be added to or inserted into a blockchain at step 120. Method 900 ends once the feeding protocol has been added to the blockchain.

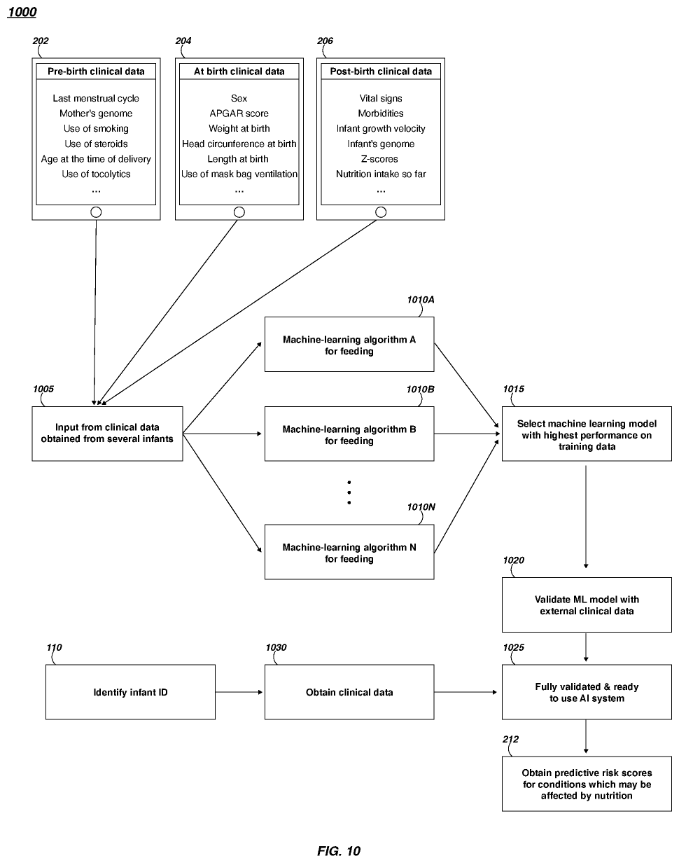

FIG. 10 illustrates an artificial intelligence method 1000 for obtaining risk scores for diseases and conditions that can be affected by nutrition including but not limited to GF, BPD, NEC, sepsis. As shown in FIG. 2 and FIG. 5, an AI server 210, which may or may not use processor 515, may receive clinical data from devices 202, 204, and 206, which may or may not be distinct devices, which includes clinical data about the infant before birth, at birth, and after birth at step 1005. This clinical data may be representative of several or multiple infants. The clinical data for these infants may be fed to a plurality of machine learning algorithms such as algorithm A 1010A, algorithm B 1010B, to algorithm N 1010n. In other words, any number of machine learning algorithms may be provided with clinical data for several infants to develop predictive ML models that output risk scores for diseases and conditions that can be affected by improved nutrition. At step 1015, the AI server 210 may select a model from algorithms A through N which has the highest performance on the provided clinical data for the several infants which was used as training data for the machine learning algorithms. The model identified at step 1015 may be validated as a machine learning (“ML”) model with new clinical data which may be external clinical data. Testing external clinical data may be used to validate the machine learning model by determining whether or not the same or similar results are obtained from untested data that were obtained by analyzing the tested data.

At step 1025, a machine learning model may be fully validated and ready to use in an artificial intelligence system implemented by AI server 210. Once an infant ID is identified at step 110, clinical data may be obtained for that particular infant at step 1030 and may be subjected to the machine learning model at step 1025. Based on this data, predictive risk scores for GF, BPD, NEC, sepsis, other diseases and conditions may be created by testing the clinical data for the particular infant using the validated machine learning model. These predictive risk scores can be instrumental in providing an infant with nutrition that is optimized to support continued growth and survival.

As discussed herein, AI server 210 may collect data over time, including pre-birth data, at birth data, and after birth data with risk factors for disease. The data may include clinical data encompassing the presence of comorbidities, drugs administered, specimen culture information, as well as vital signs taken from sensors and ventilators, ultrasounds, and related scores, and metabolic data. The data may further include nutritional data such as macronutrient and or micronutrient intake and fortifiers administered. The data may further include growth data including weight data, length data, and head circumference data. The data may further include genetic data and microbiome data for both a mother and an infant. AI server 210 may use each element of this data to produce learning algorithms that learn a function that best maps the relationship between clinical variables and a response in terms of disease risk scores and infant growth. In this manner, AI server 210 will enable optimal personalized nutrition, including milk fortification, for infants. Furthermore, AI server 210 may transmit disease risk scores to device 206 for presentation to healthcare professionals. The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A system, comprising:
a sensor or set of sensors which analyze a sample of human milk, and
an artificial intelligence server including a processor or several processors and an artificial intelligence engine which:
receives sensor data generated by the sensor or sensors;
analyzes the sensor data to identify concentrations of macronutrients and or micronutrients in the sample of milk;
compares the concentrations of macronutrients and or micronutrients in the sample of human milk with nutritional guidelines for a particular infant;
compares the concentrations of macronutrients and or micronutrients in the sample of human milk with feeding protocols obtained from historical clinical data about one or more infants with similar clinical profiles to the particular infant;
identifies one or more disease risk scores based on clinical data specific to the particular infant; and
provides to a device a nutritional recommendation for optimal personalized milk fortification based on the comparison of the macronutrients and or micronutrients in the sample of milk with chosen nutritional guidelines, and with feeding protocols obtained from historical clinical data about one or more infants with similar clinical profiles for the particular infant, and optionally disease risk scores.

2. The system of claim 1, wherein the sensor or sensors are spectrometers, spectrographs, or other suitable sensors.

3. The system of claim 1, wherein the optimized nutrition recommendation is specific to the particular infant and the particular infant has a birthweight less than 5 pounds, 8 ounces.

4. The system of claim 1, wherein the clinical data may include disease risk score information about the particular infant.

5. The system of claim 4, wherein the clinical data include a risk score of the particular infant to contract a disease.

6. The system of claim 5, wherein the optimized nutritional recommendation reflects the one or more disease risk scores.

7. The system of claim 4, wherein the one or more disease risk scores include clinical data information about the pre-birth period, at-birth period, or post-birth period of the particular infant.

8. A method, comprising:
receiving, by an artificial intelligence server which includes one or more processors, sensor data generated by a sensor or sensors;
analyzing, by the artificial intelligence server which includes one or more processors, the sensor data to identify concentrations of macronutrients and or micronutrients, contamination, and or freshness of a sample of milk;
comparing, by the artificial intelligence server which includes one or more processors, the concentrations of macronutrients and or micronutrients in the sample of milk with chosen nutritional guidelines;
comparing, by the artificial intelligence server which includes one or more processors, the concentrations of macronutrients and or micronutrients in the sample of human milk with feeding protocols obtained from historical clinical data about one or more infants with similar clinical profiles to the particular infant;
identifying, by the artificial intelligence server which includes one or more processors, one or more risk scores for a particular infant based on the likelihood of developing diseases affected by nutrition, including but not limited to growth faltering, bronchopulmonary dysplasia, necrotizing enterocolitis, and sepsis; and
providing to a device, by the artificial intelligence server which includes one or more processors, an optimized personalized nutritional recommendation for milk fortification, and optionally disease risk scores.

9. The method of claim 8, further comprising:
receiving, by the artificial intelligence server that includes one or more processors, an indication that the optimized nutritional recommendation for milk fortification has been manually adjusted.

10. The method of claim 9, wherein based on the manual adjustment, the artificial intelligence server that includes one or more processors, updates the optimized nutritional recommendation for milk fortification for subsequent recommendations for the particular infant.

11. The method of claim 10, further comprising receiving, by the artificial intelligence server that includes one or more processors, feeding feedback.

12. The method of claim 11, wherein the feeding feedback indicates an amount of milk effectively consumed by the particular infant.

13. The method of claim 11, wherein the feeding feedback is correlated by the artificial intelligence server with growth of the infant over a period of time.

14. The method of claim 11, wherein the artificial intelligence server suggests the presence of an ailment or disease in the particular infant based on the feeding feedback.

15. The method of claim 11, wherein the artificial intelligence server uses both the clinical data specific to the particular infant and feeding feedback to determine a risk score for the likelihood of developing diseases or conditions which may be affected by nutrition, including but not limited to growth faltering, bronchopulmonary dysplasia, necrotizing enterocolitis, and sepsis, for a particular infant.

16. The method of claim 8, wherein the optimized nutrition recommendation for milk fortification provided to a device is generated using machine learning.

17. The method of claim 16, wherein the artificial intelligence server that includes one or more processors further updates the optimized nutrition recommendation for milk fortification based on outcomes of other infants and based on at least one shared clinical data point between the other infants and the particular infant.

18. The method of claim 17, wherein the artificial intelligence server that includes one or more processors transmits the optimized nutritional recommendation for milk fortification to the device for graphical or textual display on the device.

19. The method of claim 8, wherein the processor provides a timestamp for feeding feedback which is correlated, by the artificial intelligence server which includes one or more processors, with infant growth, and wherein the correlation is provided graphically or textually to a device.

20. A method, comprising:
receiving, by an artificial intelligence server which includes which includes one or more processors, sensor data generated by a sensor or more than one sensor;
analyzing, by the artificial intelligence server which includes one or more processors, the sensor or sensors data to identify concentrations of macronutrients and or micronutrients in a sample of milk;
comparing, by the artificial intelligence server which includes one or more processors, the constituent elements of macronutrients and micronutrients in the sample of milk with the chosen nutritional guidelines;
identifying, by the artificial intelligence server which includes one or more processors, one or more disease risk scores to a particular infant based on one or more clinical data associated with information about the particular infant and his or her parents; and
providing to a device, by the artificial intelligence server which includes one or more processors, an optimized nutritional recommendation for milk fortification.
